SEQUENCE LISTING (14 sequences)

SEQ ID NO: 1 - DNA, 1652 bp, Cocal virus
aacagtgatcaactgttccagattcaacatgaatttcctactcttgacatttattgtgttgccgttgtgcagccacgccaagttctccattgtattccctcaaagccaaaaaggcaattggaagaatgtaccatcatcttaccattactgcccttcaagttcggatcaaaactggcacaatgatttgcttggaatcacaatgaaagtcaaaatgcccaaaacacacaaagctattcaagcagacgggtggatgtgtcatgctgccaaatggatcactacctgtgactttcgctggtacggacccaaatacatcactcactccattcattccatccagcctacttcagagcagtgtaaagaaagcatcaagcaaacaaaacaaggtacttggatgagtcctggcttccctccacagaactgcgggtatgcaacagtaacagactctgtcgctgttgtcgtccaagccactcctcatcatgtcttggttgatgaatatactggagaatggatcgactctcaattccccaacgggaaatgtgaaaccgaagagtgcgagaccgtccacaactctaccgtatggtactctgactacaaagtaactggattatgtgacgcaactctggtagacacagagatcaccttcttctctgaagatggcaaaaaagaatctatcgggaagcccaacacaggctataggagcaactacttcgcttatgagaaaggggacaaagtatgtaaaatgaactactgcaagcatgcgggtgtgaggttgccttccggggtttggtttgagtttgtggatcaggatgtctacgccgccgccaaacttccagaatgccccgttggtgccactatctccgctccgacacagacctctgttgacgtaagtctcattctagatgtagagagaattttagattactctctgtgtcaagagacatggagcaagatccggtccaaacagccagtatcccctgttgaccttagttacttggcccccaagaatcctgggaccggaccggcattcacaatcatcaatggcactctgaagtactttgagaccagatacattcggattgatatagacaatccaatcatctccaagatggtggggaaaataagtggcagtcaaacagaacgagaattgtggacagagtggttcccctacgagggtgtcgagatagggccaaatgggattctcaaaacccctacaggatacaaattcccactcttcatgataggacacgggatgctagattccgacttgcacaagacgtcccaagcagaggtctttgaacatcctcaccttgcagaagcaccaaagcagttgccggaggaggagactttattttttggtgacacaggaatctccaaaaatccggtcgaactgattgaagggtggtttagtagttggaagagcactgtagtcacctttttctttgccataggagtatttatactactgtatgtagtggccagaattgtgatcgcagtgagatacagatatcaaggctcaaataacaaaagaatttacaatgatattgagatgagcagatttagaaaatgatgaagaccctcagatgattatacatatgtgatatgaaaaaaactaacagtcatcatggacttgaatgacttcgagttgagacagt

SEQ ID NO: 2 - protein, 512 aa, Cocal Virus
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 3 - DNA, 1539 bp, Artificial Sequence
  Nucleotide Sequence of the human codon enriched Cocal vesiculovirus envelope glycoprotein
atgaattttcttctcttgacctttatcgtccttccgctctgcagtcacgctaaattttcgatcgtcttcccacagagtcagaagggcaattggaagaatgtaccgagttcatatcattattgtccaagcagctctgatcaaaactggcataacgacctgctgggcattaccatgaaggtgaaaatgcctaagacacataaggcgattcaggcagacgggtggatgtgccacgcagccaagtggattacaacttgtgacttccgatggtacggtcctaagtatattactcactccatacacagcatccagcccaccagtgagcagtgcaaagagagtatcaagcagaccaagcagggaacctggatgtcacctggctttccacctcagaattgtggctatgcaacagtgacagactcagtggctgttgtggtgcaggcaaccccccaccacgtactcgttgacgaatatacaggcgaatggattgactcccagtttcccaacggtaaatgcgagacagaagagtgcgagactgtgcacaattcaacagtgtggtactccgattataaggttaccgggctttgcgacgccacactggtggacacagagataacgtttttttccgaagacggaaaaaaggaaagtatcgggaaacccaacactggataccggagcaattacttcgcgtatgagaaaggagataaggtctgcaaaatgaattattgcaaacacgccggggtaaggctgccctccggcgtgtggtttgagttcgtggaccaggacgtctacgcagccgccaaattgcccgagtgtccagtgggagctacaatttccgcaccgacacaaacctcagtggatgtgtctctgattctggacgtagagaggatcctcgactactctttgtgtcaggagacgtggagcaagatacggtctaagcaaccagtctcacccgtagatttgagctacctcgccccgaaaaacccaggcacgggcccagcgttcacgatcatcaacggcacgcttaaatatttcgagactcgctatatccgcatcgacatcgacaatcctatcatctctaagatggtgggtaagatctctggatcccagactgaacgagaactgtggacagaatggttcccctacgagggcgtcgagattggccctaacggaatactgaagacccctaccggctataagttccctctgtttatgatcggccacggaatgctggattctgatttgcataagacttcacaagcagaagtctttgaacatcctcacctcgccgaagcacctaaacagttgcctgaggaagagaccctgttcttcggcgatacaggtatatccaaaaacccggtggagcttatcgaaggttggtttagcagctggaagtcaacagtggtaactttcttcttcgccatcggcgtgtttatacttctgtacgtagtggcccgcatcgtgatcgcagtgcgctacagataccaaggaagcaacaacaaaagaatctacaacgacatagagatgagccgcttcaggaagtga

SEQ ID NO: 4 - protein, 512 aa, Artificial Sequence; identical to SEQ ID NO: 2
  Amino acid sequence of human codon enriched Cocal vesiculovirus envelope glycoprotein

SEQ ID NO: 5 - DNA, 5722 bp, Artificial Sequence
  Sequence of Cocal vector plasmid pMD2.CocalG
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcatcgatgcggccgccaccatgaattttcttctcttgacctttatcgtccttccgctctgcagtcacgctaaattttcgatcgtcttcccacagagtcagaagggcaattggaagaatgtaccgagttcatatcattattgtccaagcagctctgatcaaaactggcataacgacctgctgggcattaccatgaaggtgaaaatgcctaagacacataaggcgattcaggcagacgggtggatgtgccacgcagccaagtggattacaacttgtgacttccgatggtacggtcctaagtatattactcactccatacacagcatccagcccaccagtgagcagtgcaaagagagtatcaagcagaccaagcagggaacctggatgtcacctggctttccacctcagaattgtggctatgcaacagtgacagactcagtggctgttgtggtgcaggcaaccccccaccacgtactcgttgacgaatatacaggcgaatggattgactcccagtttcccaacggtaaatgcgagacagaagagtgcgagactgtgcacaattcaacagtgtggtactccgattataaggttaccgggctttgcgacgccacactggtggacacagagataacgtttttttccgaagacggaaaaaaggaaagtatcgggaaacccaacactggataccggagcaattacttcgcgtatgagaaaggagataaggtctgcaaaatgaattattgcaaacacgccggggtaaggctgccctccggcgtgtggtttgagttcgtggaccaggacgtctacgcagccgccaaattgcccgagtgtccagtgggagctacaatttccgcaccgacacaaacctcagtggatgtgtctctgattctggacgtagagaggatcctcgactactctttgtgtcaggagacgtggagcaagatacggtctaagcaaccagtctcacccgtagatttgagctacctcgccccgaaaaacccaggcacgggcccagcgttcacgatcatcaacggcacgcttaaatatttcgagactcgctatatccgcatcgacatcgacaatcctatcatctctaagatggtgggtaagatctctggatcccagactgaacgagaactgtggacagaatggttcccctacgagggcgtcgagattggccctaacggaatactgaagacccctaccggctataagttccctctgtttatgatcggccacggaatgctggattctgatttgcataagacttcacaagcagaagtctttgaacatcctcacctcgccgaagcacctaaacagttgcctgaggaagagaccctgttcttcggcgatacaggtatatccaaaaacccggtggagcttatcgaaggttggtttagcagctggaagtcaacagtggtaactttcttcttcgccatcggcgtgtttatacttctgtacgtagtggcccgcatcgtgatcgcagtgcgctacagataccaaggaagcaacaacaaaagaatctacaacgacatagagatgagccgcttcaggaagtgaaggcctgaattcaccccaccagtgcaggctgcctatcagaaagtggtggctggtgtggctaatgccctggcccacaagtttcactaagctcgcttccttgctgtccaatttctattaaaggttccttggttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagtttggctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 6 - DNA, 32 bp, Artificial Sequence
  Primer Hygromycin-F-XhoI
gagccctcgagatgaaaaagcctgaactcacc

SEQ ID NO: 7 - DNA, 40 bp, Artificial Sequence
  Primer Hygromycin-R-2A
agacttcctctgccctctgagttagcctcccccatctccc

SEQ ID NO: 8 - DNA, 39 bp, Artificial Sequence
  Prime T2A-F-Hyg
gggagatgggggaggctaactcagagggcagaggaagtc

SEQ ID NO: 9 - DNA, 43 bp, Artificial Sequence
  Primer T2A-R-VSVG
ctaagtacaaaaggcacttcatggggcccggattctcctccac

SEQ ID NO: 10 - DNA, 42 bp, Artificial Sequence
  Primer T2A-R-cocal
ggtcaagagaagaaaattcatggggcccggattctcctccac

SEQ ID NO: 11 - DNA, 41 bp, Artificial Sequence
  Primer Cocal-F-2A
tggaggagaatccgggccccatgaattttcttctcttgacc

SEQ ID NO: 12 - DNA, 33 bp, Artificial Sequence
  Primer Cocal-R-HindIII
gagccaagctttcacttcctgaagcggctcatc

SEQ ID NO: 13 - DNA, 42 bp, Artificial Sequence
  Primer VSVG-F-2A
tggaggagaatccgggccccatgaagtgccttttgtacttag

SEQ ID NO: 14 - DNA, 33 bp, Artificial Sequence
  Primer VSVG-R-HindIII
gagccaagcttttactttccaagtcggttcatc

(19) United States
(12) Patent Application Publication
(10) Pub. No.: US 2018/0127781 A1
(43) Pub. Date: May 10, 2018

(54) COCAL ENVELOPE PSEUDOTYPED RETROVIRAL VECTOR PRODUCER CELLS

(71) Applicant: FRED HUTCHINSON CANCER RESEARCH CENTER, Seattle, WA (US)

(72) Inventors: Hans-Peter Kiem, Seattle, WA (US); Olivier Humbert, Seattle, WA (US)

(21) Appl. No.: 15/545,582

(22) PCT Filed: Jan. 21, 2016
(86) PCT No.: PCT/US16/14367

§ 371 Date: Jul. 21, 2017

Related U.S. Application Data
(60) Provisional application No. 62/106,184, filed on Jan. 21, 2015.

(51) Int. Cl. C12N 15/867; C12N 5/071; C12N 5/073; C12N 7/02

(52) U.S. Cl. CPC C12N 15/867; C12N 5/0686; C12N 5/0603; C12N 2740/16043; C12N 2740/16045; C12N 2740/16051; C12N 2810/6081; C12N 7/025

(57) ABSTRACT

Producer cell lines that produce cocal envelope pseudotyped retroviral vectors are described. The producer cells can be grown and can produce the cocal envelope pseudotyped retroviral vectors in large scale serum-free suspensions.

### CROSS REFERENCE TO RELATED APPLICATION

The present application claims the benefit of U.S. Provisional Patent Application No. 62/106,184 filed Jan. 21, 2015, which is incorporated herein by reference in its entirety as if fully set forth herein.

### STATEMENT OF GOVERNMENT SUPPORT

This invention was made with government support under grants AI097100, DK056465, and HL116217 awarded by the National Institutes of Health. The government has certain rights in the invention.

### FIELD OF THE DISCLOSURE

The present disclosure provides producer cell lines that produce cocal envelope pseudotyped retroviral vectors. The producer cells can be grown and can produce the cocal envelope pseudotyped retroviral vectors in large scale serum-free suspensions.

### BACKGROUND OF THE DISCLOSURE

Lentiviral vectors (LVs) are currently considered the gold standard for hematopoietic stem cell (HSC) gene therapy and for immunotherapies with genetically modified T cells. These vectors were first developed in the early 1990s and are typically made by transient transfection of helper and vector plasmids into cells that support the assembly of LV virions. LVs have commonly been pseudotyped with the heterologous vesicular stomatitis virus envelope glycoprotein (VSV-G), which confers broad tropism and stability to the vector. However, VSV-G is inactivated by human serum complement, making it unsuitable for in vivo delivery, and is cytotoxic when stably expressed in human cells, which has impeded efforts to develop LV producer cell lines.

A cocal vesiculovirus envelope glycoprotein to pseudotype LV has been developed. The cocal envelope glycoprotein shares 71.5% identity at the amino acid level with the VSV-G Indiana envelope, and cocal pseudotyped LVs (coca) LVs) were found to have broad tropism and to be more resistant to inactivation by human serum than VSV-G pseudotyped LVs (VSV-G LVs). In addition, cocal LVs can be produced at high titers and efficiently transduce human, nonhuman primate, and canine hematopoietic stem cells.

### SUMMARY OF THE DISCLOSURE

The current disclosure describes the development of high titer self-inactivating (SIN) LV producer cell lines based on the cocal envelope. Lentivirus producer cell lines that stably express all the different components required for the assembly of LV have several advantages over traditional production methods that use transient transfection of plasmids: 1) reproducibility and consistency in vector titer and quality; 2) safety: the absence of DNA in the preparation avoids the risk of recombination between transfected plasmids and the production of replication-competent lentiviruses; 3) cost: clinical grade plasmid DNA is expensive and considerably adds to the cost of the vector; and 4) scale-up: producer cells can be adapted to grow in suspension cultures suitable for bioreactors.

### BRIEF DESCRIPTION OF THE FIGURES

FIG. 1A-B (FIG. 1A) Schematic representation of cocal and VSV-G envelope plasmids and lentiviral transfer plasmids used for the generation of LV producer cells. (FIG. 1B) Unconcentrated LV titer resulting from standard LV production by transient transfection (left), or from cells stably expressing each envelope (right). Results are given as infectious units per mL (IU/mL) and show means from one representative experiment. Error bars show standard error of the mean. CMV=human cytomegalovirus promoter. hBGint=human beta globin intronic sequence. 2A=self-cleaving peptide sequence. hBpA=human beta globin polyA sequence. ΔLTR=long terminal repeat containing short deletion. PGK=phosphoglycerate kinase 1 promoter (human). eGFP=enhanced green fluorescent protein. wpre=woodchuck hepatitis virus regulatory element. MSCV=murine stem cell virus promoter. TCRα=T cell receptor alpha chain. TCRβ=T cell receptor beta chain.

FIG. 2A-E (FIG. 2A) Transduction efficiency of nonhuman primate (NHP) CD34⁺ cells (n=1) exposed to LVs at an MOI of 2×5. The percentage of eGFP+ cells was determined at 10 days post transduction and error bars show standard error of the mean. (FIG. 2B) Mean transduction efficiency of human CD34⁺ cells from two different donors exposed to LVs at an MOI of 5. The percentage of eGFP+ cells was determined 6 days post transduction and error bars show standard error of the mean. (FIG. 2C) Human CD34⁺ cells from one of the donor described in (FIG. 2B) were plated for colony-forming cells (CFCs). The fraction of progenitor cells for different lineages was enumerated and compared between mock, VSV-G or cocal LV transduction. E:Erythroid, M:Monocyte, GM:Granulocyte/Macrophage, GEMM:Granulocyte/Erythrocyte/Macrophag e/Megakaryocyte. (FIG. 2D) Transduction efficiency in nonhuman primate peripheral blood from 2 donors enriched for CD4⁺, activated for 3 days and exposed to LVs at an MOI of 5. The percentage of eGFP+ cells was determined at 3 days post transduction. (FIG. 2E) Transduction efficiency in human CD4⁺ cells exposed to 2 different LV preparations (LV1 and LV2) at an MOI of 1. The percentage of eGFP+ cells was determined at 3 days post transduction.

FIG. 3. Interference assay to look at receptor specificity for the cocal and VSV-G envelopes.

FIG. 4A-D. (FIG. 4A) Diagram highlighting the steps and plasmids used toward the generation of LV producer cell lines. Stable expression was achieved by DNA transfection followed by selection with the respective drugs. (FIG. 4B) LV titer determined by flow cytometry for single cocal and VSV-G packaging clones (open circles) or from bulk cells (grey bar). Unconcentrated titer is given as infectious units/mL (IU/mL). (FIG. 4C) Unconcentrated (1×) and concentrated (100×) LV titers obtained from the best producer cocal or VSV-G clones identified in (B) grown in 15-cm plates. Results are means from one representative experiment. Error bars show standard deviations. (FIG. 4D) Stability of cocal and VSV-G packaging cell lines as determined by unconcentrated titers produced following long-term culture.

FIG. 5. Induction of titer in LV packaging cells with sodium butyrate.

FIG. 6A-F. (FIG. 6A) Flow cytometry analysis of cocal producer cells expressing varying levels of eGFP fluorescence, represented by flow chart (left) or histogram (right). (FIG. 6B) Sorting of cocal producer cells from (FIG. 6A) based on eGFP fluorescence intensity (histogram) and corresponding LV titer measured in each subpopulation (bar chart). (FIG. 6C) LV titer measured in single eGFP producer clones (open circles) or bulk cells (bar) after the 1^{st} or 2^{nd} round of screening. Unconcentrated titer is given as infectious units/mL (IU/mL). (FIG. 6D) Unconcentrated (1×) and concentrated (100×) LV titers from the best eGFP producer clone identified in (FIG. 6C) from the first screen. Bar graph shows mean titer from one representative experiment and error bars show standard error of the mean. (FIG. 6E) Stability of eGFP LV producer cell line based on unconcentrated titer after long-term culture. (FIG. 6F) Transduction of nonhuman primate CD34⁺ cells by LV generated with the standard protocol (black) or using producer cells (grey). Different multiplicities of infection (MOIs) of vector were used and cells were analyzed by flow cytometry at 4 days post transduction.

FIG. 7A-7F. (FIG. 7A) LV titer measured by qPCR in single cocal TCR producer clones (open circles) or bulk cells (bar). Unconcentrated titer is given as infectious units/mL (IU/mL). (FIG. 7B) Unconcentrated (1×) and concentrated (100×) LV titer measured by qPCR from multiple experiments (n=5) using the best producer clone identified in (FIG. 7A). Bar graph shows mean titer and error bars show standard error of the mean. (FIG. 7C) Stability of cocal TCR LV producer cell line based on unconcentrated titer after long-term culture. (FIG. 7D) TCR expression measured in H9 cell line transduced with different amounts of LV produced with the standard protocol or with the producer cell line. TCR expression was detected by surface antibody staining at 3 days post transduction and the fraction of TCR positive cells was quantified by flow cytometry. (FIG. 7E) TCR expression measured in human CD4⁺ cells transduced with different amounts of vector as described in (FIG. 7D). (FIG. 7F) Histogram showing levels of TCR expression measured in human CD4⁺ cells from (FIG. 7E) transduced with 5 μL LV made with producer cells (grey line) or with standard conditions (black line), as compared to unstained cells (grey filled).

FIG. 8. Second round of screening for best producer clones for TCR producer cells.

FIG. 9A-9C. (FIG. 9A) Growth rate of 293T cells, eGFP and TCR LV producer cells adapted to growth in suspension/serum free media. Cells were passaged every 3 to 4 days, and viable count was determined using a hemocytometer by trypan blue staining. (FIG. 9B) Unconcentrated LV titer measured by flow cytometry in eGFP LV producer cells grown in suspension over time. (FIG. 9C) Unconcentrated LV titer measured by qPCR from suspension TCR LV producer cells grown in suspension over time.

FIG. 10. Nucleotide sequence of Cocal vesiculovirus envelope glycoprotein (GenBank Accession No. AF045556; SEQ ID NO: 1) and amino acid sequence of Cocal vesiculovirus envelope glycoprotein (SEQ ID NO: 2) encoded by the nucleotide sequence of FIG. 10 (SEQ ID NO: 1).

FIG. 11. Nucleotide sequence of the human codon enriched Cocal vesiculovirus envelope glycoprotein (SEQ ID NO: 3) based on the nucleotide sequence of FIG. 10 (SEQ ID NO: 1) and amino acid sequence of human codon enriched Cocal vesiculovirus envelope glycoprotein (SEQ ID NO: 4) encoded by the nucleotide sequence of FIG. 12 (SEQ ID NO: 3).

FIG. 12. Nucleotide sequence of the lentiviral vector designated pMD2.CocalG (SEQ ID NO: 5).

### DETAILED DESCRIPTION

Lentiviral vectors (LVs) are currently considered the gold standard for hematopoietic stem cell (HSC) gene therapy and for immunotherapies with genetically modified T cells. These vectors were first developed in the early 1990s and are typically made by transient transfection of helper and vector plasmids into cells that support the assembly of LV virions. LVs have commonly been pseudotyped with the heterologous vesicular stomatitis virus envelope glycoprotein (VSV-G), which confers broad tropism and stability to the vector. However, VSV-G is inactivated by human serum complement, making it unsuitable for in vivo delivery, and is cytotoxic when stably expressed in human cells, which has impeded efforts to develop LV producer cell lines.

Cocal vesiculovirus envelope proteins have been described in the literature (see, for example, Bhella et al., Virus Res. 54:197-205 (1998) and GenBank Accession No. AF045556), and cocal vesiculovirus envelope glycoproteins to pseudotype LV have been developed. The cocal envelope glycoprotein shares 71.5% identity at the amino acid level with the VSV-G Indiana envelope, and cocal pseudotyped LVs (cocal LVs) were found to have broad tropism and to be more resistant to inactivation by human serum than VSV-G pseudotyped LVs (VSV-G LVs). In addition, cocal LVs can be produced at high titers and efficiently transduce human, nonhuman primate, and canine hematopoietic stem cells.

Disclosed herein is the development of two high-titer, 3^{rd} generation, LV producer cell lines employing the cocal envelope for a standard eGFP transgene and for a TCR specific for the tumor antigen WT1. Lentivirus producer cell lines that stably express all the different components required for the assembly of LV have several advantages over traditional production methods that use transient transfection of plasmids: 1) reproducibility and consistency in vector titer and quality; 2) safety: the absence of DNA in the preparation avoids the risk of recombination between transfected plasmids and the production of replication-competent lentiviruses; 3) cost: clinical grade plasmid DNA is expensive and considerably adds to the cost of the vector; and 4) scale-up: producer cells can be adapted to grow in suspension cultures suitable for bioreactors.

The use of the cocal envelope was a key component for successfully achieving high-titer LV because stable expression of the commonly used VSV-G envelope led to significantly lower titers. Additional advantages of cocal LVs include resistance to human serum inactivation, and higher transduction efficiency of human and NHP HSCs and CD4⁺ T cells at low vector doses. Indirect evidence of shared receptor usage by cocal and VSV-G LV is also provided, shown to be the highly ubiquitous low-density lipoprotein (LDL) receptor for the VSV-G glycoprotein. Finkelshtein et al., Proc Natl Acad Sci USA 110: 7306-7311 (2013). This result is consistent with the high amino acid sequence conservation shared by the two envelope proteins and implies that cocal and VSV-G LVs use similar uptake and internalization pathways for cell entry.

The generation of a high-titer cocal packaging cell line provides a great foundation for the construction of additional LV producer cell lines, by stable expression of a lentiviral transfer plasmid and selection of best producer single clones, which should be completed within 2 months. A widely used standard eGFP lentiviral plasmid was first used to streamline and develop the described protocols, which were then applied to the development of a WT1-specific TCR LV cell line. It was found that LV generated by producer cells performed equally well, if not better, than LV made with more standard protocols for the transduction of primary cells. This could be the result of a higher ratio of infectious particles to nonfunctional or immature particles generated by producer cells.

The histone deacetylase (HDAC) inhibitor sodium butyrate was used to achieve high LV titers and thus efficient transduction of HSCs and T cells. Previous work showed that inhibition of HDACs by sodium butyrate increases LV titer using a transient transfection protocol. Ellis, et al., Hum Gene Ther 22: 93-100 (2011); Olsen, et al., Hum Gene Ther 6: 1195-1202 (1995). Without being bound by theory, this induction in LV titer can probably be accounted for, at least in part, by an increase in steady-state levels of viral vector RNA within the producer cells. In the absence of sodium butyrate treatment, LV production is kept at minimal levels, allowing producer cells to grow robustly with limited toxicity associated with the expression of viral proteins. Other exemplary HDAC inhibitors that can be used include Scriptaid, APHA Compound 8, Apicidin, (−)-Depudecin, Sirtinol, and trichostatin A.

In current gene therapy HSC trials, roughly 1 to 5×10⁹ IU of vector per patient is required. Based on our LV titers, this would necessitate the production of 0.5 L to 2.5 L μg unconcentrated vector per patient. While production of such LV preparation is certainly feasible using adherent producer cells, the use of suspension producer cells would greatly facilitate this process, especially with cultures grown in bioreactors. To this end, it is shown that the disclosed producer cells were adapted to grow in suspension, serum-free media and LV was produced using a 1 L bioreactor. Although LV titers obtained from these cells are currently 3 to 5-fold lower than those obtained with adherent cells, culture parameters will further be developed to increase LV production.

The broad applicability of the disclosed cocal packaging cell lines offer a promising tool toward efforts aimed at generating large-scale, clinical grade LV.

Stable expression refers to consistent expression within a range (e.g., 1.00E+06-1.00E+07 for at least 100 days or at least 120 days). Stable expression can also be higher levels of expression than 1.00E+06-1.00E+07 for more than 120 days.

The producer cells disclosed herein can utilize plasmid vectors for the expression of Cocal vesiculovirus envelope proteins, which vectors include a polynucleotide encoding a Cocal vesiculovirus envelope protein wherein the polynucleotide is under the transcriptional control of a eukaryotic transcriptional promoter. Exemplary polynucleotides that encode a Cocal vesiculovirus envelope protein include the nucleotide sequence of SEQ ID NO: 1 (GenBank Accession No. AF045556), which encodes the protein having the amino acid sequence of SEQ ID NO: 2, as well as a human codon-optimized variant of that nucleotide sequence that is presented as SEQ ID NO: 3 and which encodes the amino acid sequence of SEQ ID NO: 4.

An exemplary plasmid vector for the expression of a Cocal vesiculovirus envelope proteins is designated herein as pMD2.CocalG, which has the nucleotide sequence presented as SEQ ID NO: 5. The pMD2.CocalG plasmid is derived from the pMD2.G described by Didier Trono and that is available from Addgene (Cambridge, Mass., Plasmid No. 12259). More specifically, the pMD2.CocalG plasmid was generated by removing the VSV-G coding sequence from the pMD2.G plasmid and ligating a polynucleotide encoding a Cocal vesiculovirus envelope protein between the human β-globin intron and polyadenylation sequences and downstream of the constitutively active CMV promoter. In the case of the pMD2.CocalG plasmid described herein, the polynucleotide encoding the Cocal vesiculovirus envelope protein was first human codon-optimized using GeneMaker technology (Blue Heron Biotechnology, Bothell, Wash.). It will be understood that this exemplary polynucleotide (SEQ ID NO: 3) is representative of a wide range of human codon-optimized polynucleotides that may be employed in the presently disclosed plasmid vectors for the expression of a Cocal vesiculovirus envelope protein.

Suitable lentiviral vector plasmids include, for example, the SIN HIV vector plasmids pRRLSIN.cPPT.PGK-GFP.WPRE (Addgene Plasmid No. 12252) and pRRLSIN.cPPT.PGK-YFP.WPRE (Naldini, San Raffaele Telethon Institute for Gene Therapy, Italy), both of which contain a central polypurine tract, a woodchuck post-transcriptional regulatory element, and an internal phosphoglycerate kinase (PGK) promoter driving expression of enhanced green fluorescent protein (EGFP) or enhanced yellow fluorescent protein (EYFP). An exemplary helper plasmid is pCMV.Δ.R8.74 described by Dull et al., J. Virol. 72:8463-8471 (1998).

The Cocal vesiculovirus envelope pseudotyped lentiviral vectors produced by producer cell lines disclosed herein can include lentiviral Gag, Pol, and one or more accessory protein(s) and a Cocal vesiculovirus envelope protein. Exemplified herein are Cocal vesiculovirus envelope pseudotyped lentiviral vector particles wherein the envelope protein (SEQ ID NO: 2) is encoded by a polynucleotide including the nucleotide sequence of SEQ ID NO: 1, as well as envelope proteins encoded by human codon-optimized nucleotide sequences such as, for example, the nucleotide sequence of SEQ ID NO: 3. One such Cocal vesiculovirus envelope protein encoded by a human codon-optimized polynucleotide is exemplified by the amino acid sequence of SEQ ID NO: 4. Cocal envelope pseudotyped lentiviral vector particles described herein typically result in concentrated titers of at least 10⁶, 10⁷, 10⁸, or 10⁹ transducing units (TU)/ml.

Producer cells disclosed herein can also produce Cocal envelope pseudotyped retroviral vector particles including alpharetroviral, betaretroviral, gammaretroviral, deltaretroviral, and epsilonretroviral vector particles.

Produced particles can be used to deliver a gene of interest to any appropriate target cell (e.g., T cells, hematopoietic cells (e.g., CD34⁺ cells)). Exemplary genes of interest include those that can be used to treat an immune-mediated condition (e.g., Grave's Disease, rheumatoid arthritis, pernicious anemia, Multiple Sclerosis (MS), inflammatory bowel disease, systemic lupus erythematosus (SLE) or severe combined immunodeficiency disease (SCID)); diseases related to red blood cells and clotting (e.g., hemoglobinopathy like thalassemia, or a sickle cell disease/trait); a lysosomal storage disorder (e.g., mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; alpha-mannsidosis; beta-mannosidosis; glycogen storage disease type I, also known as GSDI, von Gierke disease, or Tay Sachs; a hyperproliferative disease (e.g., cancer); an infectious disease (e.g., HIV, malaria). Particular examples, include gene or gene products such as soluble CD40, CTLA, Fas L, antibodies to CD4, CD5, CD7, CD52, etc., antibodies to IL1, IL2, IL6, an antibody to TCR specifically present on autoreactive T cells, IL4, IL10, IL12, IL13, IL1Ra, sIL1RI, sIL1RII, sTNFRI, sTNFRII, antibodies to TNF, P53, PTPN22, DRB1*1501/DQB1*0602, HBB, CYB5R3, F8, F9, IDUA or iduronidase, IDS, GNS, HGSNAT, SGSH, NAGLU, GUSB, GALNS, GLB1, ARSB, HYAL1, 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, α2β1, αvβ3, αvβ5, αvβ63, BOB/GPR15, Bonzo/STRL-33/TYMSTR, CCR2, CCR3, CCR5, CCR8, CD4, CD46, CD55, CXCR4, aminopeptidase-N, HHV-7, ICAM, ICAM-1, PRR2/HveB, HveA, α-dystroglycan, LDLR/α2MR/LRP, PVR, PRR1/HveC, laminin receptor, erythrocyte skeletal protein 4.1, glycophorin, p55, and the Duffy allele.

### Exemplary Embodiments

1. A producer cell that stably expresses a cocal envelope, retroviral helpers and a retroviral vector.
2. A producer cell of embodiment 1 wherein the producer cell additionally expresses a selection marker.
3. A producer cell of embodiment 1 or 2 wherein the producer cell additionally expresses a self-cleaving peptide.
4. A producer cell of any of embodiments 1-3 wherein the producer cell stably expresses the cocal envelope, retroviral helpers and retroviral vector for at least 100 days.
5. A producer cell of any of embodiments 1-4 wherein the producer cell stably expresses the cocal envelope, retroviral helpers and retroviral vector while in suspension.
6. A producer cell of embodiment 5 wherein the suspension is serum-free.
7. A producer cell of embodiment 4 or 5 wherein the suspension is at least 1 liter.
8. A producer cell of any of embodiments 1-7 wherein the retroviral vector encodes a therapeutic protein.
9. A producer cell of any of embodiments 1-8 wherein the retroviral vector includes or encodes soluble CD40, CTLA, Fas L, antibodies to CD4, CD5, CD7, CD52, etc., antibodies to IL1, IL2, IL6, an antibody to TCR specifically present on autoreactive T cells, IL4, IL10, IL12, IL13, IL1Ra, sIL1RI, sIL1RII, sTNFRI, sTNFRII, antibodies to TNF, P53, PTPN22, DRB1*1501/DQB1*0602, HBB, CYB5R3, F8, F9, IDUA or iduronidase, IDS, GNS, HGSNAT, SGSH, NAGLU, GUSB, GALNS, GLB1, ARSB, HYAL1, 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, α2β1, αvβ3, αvβ5, αvβ63, BOB/GPR15, Bonzo/STRL-33/TYMSTR, CCR2, CCR3, CCR5, CCR8, CD4, CD46, CD55, CXCR4, aminopeptidase-N, HHV-7, ICAM, ICAM-1, PRR2/HveB, HveA, α-dystroglycan, LDLR/α2MR/LRP, PVR, PRR1/HveC, laminin receptor, erythrocyte skeletal protein 4.1, glycophorin, p55, and the Duffy allele
10. A producer cell of any of embodiments 1-9 wherein the producer cell is a human embryonic kidney (HEK) 293 cell.
11. A producer cell of any of embodiments 1-10 wherein the stable expression of the cocal envelope, retroviral helpers and retroviral vector results in production of a cocal envelope pseudotyped retroviral vector.
12. A method of producing a producer cell that stably produces a cocal envelope pseudotyped retroviral vector including:

Transfecting a cell with a plasmid encoding a cocal envelope, a self-cleaving peptide, and a selection marker;

Transfecting the cell with one or more helper plasmids when the cell is stably expressing the cocal envelope; and

Transfecting the cell with a retroviral plasmid when the cell is stably expressing the cocal envelope.
13. A method of embodiment 12 wherein the retroviral plasmid is a lentiviral plasmid.
14. A method of embodiment 12 or 13 wherein the producer cell is a human embryonic kidney (HEK) 293 cell.
15. A method of harvesting a cocal envelope pseudotyped retroviral vector produced by a cell of claim 1 comprising:

washing a cell of claim 1;

treating the cell with sodium butyrate;

harvesting the cell's supernatant;

filtering the supernatant; and

centrifuging the filtrate.
16. A method of embodiment 15 wherein sodium butyrate is replaced or supplemented with Scriptaid, APHA Compound 8, Apicidin, (−)-Depudecin, Sirtinol, or trichostatin A.

### EXAMPLES

Materials and Methods. Plasmids. pMD2.G, pMDLg/pRRE, and pRSV-Rev were gifts from Didier Trono (Addgene plasmid # 12259, 12251, 12253, Cambridge, Mass.). Dull, et al., J Virol 72: 8463-8471 (1998). The Hygromycin.2A.cocal and Hygromycin.2A.VSV-G cassettes were derived from plasmids pTK-Hyg (Clontech Laboratories, Inc., Mountain View, Calif.), pMD2.G, and pMD2.Cocal-G (Trobridge et al., Mol Ther 18: 725-733 (2010)) and assembled by overhang PCR using High Fidelity Platinum Taq polymerase (Invitrogen Corp, Carlsbad, Calif.) and subcloned into the Xhol/HindIII restriction sites of plasmid Bluescript SK+ (Stratagene California, La Jolla, Calif.). Primer sequences provided in the following Table 1.

TABLE 1
Primer Sequences

| Primer name | Primer sequence |
|---|---|
| Hygromycin-F- | GAGCCCTCGAGATGAAAAAGCCTGAACTCACC |
| XhoI | (SEQ ID NO: 6) |
| Hygromycin-R- | AGACTTCCTCTGCCCTCTGAGTTAGCCTCCCCCA |
| 2A | TCTCCC (SEQ ID NO: 7) |
| T2A-F-Hyg | GGGAGATGGGGGAGGCTAACTCAGAGGGCAGAGG |
| | AAGTC (SEQ ID NO: 8) |
| T2A-R-VSVG | CTAAGTACAAAAGGCACTTCATGGGGCCCGGATT |
| | CTCCTCCAC (SEQ ID NO: 9) |
| T2A-R-cocal | GGTCAAGAGAAGAAAATTCATGGGGCCCGGATTC |
| | TCCTCCAC (SEQ ID NO: 10) |
| Cocal-F-2A | TGGAGGAGAATCCGGGCCCCATGAATTTTCTTCT |
| | CTTGACC (SEQ ID NO: 11) |
| Cocal-R-HindIII | GAGCCAAGCTTTCACTTCCTGAAGCGGCTCATC |
| | (SEQ ID NO: 12) |
| VSVG-F-2A | TGGAGGAGAATCCGGGCCCCATGAAGTGCCTTTT |
| | GTACTTAG (SEQ ID NO: 13) |
| VSVG-R-HindIII | GAGCCAAGCTTTTACTTTCCAAGTCGGTTCATC |
| | (SEQ ID NO: 14) |

Integrity of the sequences was confirmed by capillary sequencing (Fred Hutch, shared resources). The inserts were then extracted with restriction enzymes Notl/Xhol (New England Biolabs (NEB), Ipswich, Mass.), Xhol was blunted with DNA pol I (NEB), and the resulting product was ligated into the Notl/Pmll sites of pMD2.Cocal-G to create pMD2.hyg.2A.cocal and pMD2.hyg.2A.VSV-G. Puromycin, Basticidin and Zeocin selections were carried out using plasmid pPUR (Clontech), pSELECT-blasti (InvivoGen, San Diego, Calif.) and pSELECT-zeo (InvivoGen), respectively. The SIN LV transfer vector pRRLSIN.cPPT.PGK-GFP.WPRE was a gift from Didier Trono (Addgene plasmid # 12252) and pRRLSIN.C4b17_P2A_aWPRE-1 was generated by cloning the alpha and beta chains of the WT1-specific TCR C4, separated by a P2A element, into pRRLSIN.cPPT.MSCV/GFP.wPRE, replacing GFP. The pRRLSIN.cPPT.MSCV/GFP. wPRE vector was a kind gift from Richard Morgan. Jones et al., Hum Gene Ther 20: 630-640 (2009).

Lentivirus and foamy virus vector production. In the standard protocol, 3^{rd} generation LVs were produced by four plasmid polyethylenimine transfection in human embryonic kidney (HEK) 293 T cells. Using 15-cm plates, cells were plated on 0.1% gelatin at a density of 1.8×10⁷ cells/plate and transfected with 27 μg transfer vector construct, 6 μg pMDLg-pRRE, 12 μg pRSC-Rev, and 6 μg pMD2.G for VSV-G pseudotyped LV or 3 μg pMD2.Cocal-G for cocal-pseudotyped LV. When 10-cm plates or 6-well plates were used to produce LV, the number of cells and amount of DNA used was scaled down proportionally to the surface area of the plate. The next day, cells were washed with 1× Dulbecco's Phosphate Buffered (Thermo Fischer Scientific, Waltham, Mass.) and treated with 15 mL media containing 10 mM sodium butyrate (Sigma-Aldrich) for 8 hours. Cell supernatant was harvested and combined with two additional harvests carried out over a time span of 48 hours. The supernatant was filtered through a 0.8 μm-pore-size filter, concentrated 100-fold by centrifugation for 15-20 hours at 4° C. at 5,000 rpm, and stored at −80° C. For LV generated in 15-cm plates using the producer cells, cells were plated on 0.1% gelatin at a density of 2.8×10⁷ cells/plate and media was replaced with 25 mL fresh media containing 10 mM sodium butyrate the next day. A single harvest was carried out at 48 hours post media change, and the resulting LV was purified and concentrated similarly to the standard protocol. Foamy virus vector was produced by polyethylenimine transfection of HEK 293T cells as previously described (Kiem et al., Gene Ther 17: 37-49 (2010)) using the transfer vector pFV.PGK.GFP.

Determination of LV titer. The titer of the vector preparations was determined by adding different amounts of LV to the human fibrosarcoma cell line HT1080 plated at 1×10⁵ cells/mL one day before transduction. Protamine sulfate was added before the addition of vector at a final concentration of 8 μg/mL. For vectors containing GFP, transduced cells were analyzed by flow cytometry 3 days post vector addition, and the percentage of GFP-expressing cells was used to calculate the number of infectious units (IU) per mL of vector. For lentiviral vectors not containing a fluorophore, titer was determined by a TaqMan assay. Briefly, HT1080 cells were transduced as described above and were passaged every 3-4 days for a total of 10 days, after which genomic DNA was extracted using the QIAamp DNA Blood Mini Kit (Qiagen N.V., Hilden, Germany). LV DNA was measured by quantitative PCR using the TaqMan assay (Applied Biosystems, Foster City, Calif.) and compared to a standard curve to determine the number of infectious units (IU) per mL.

Culture and transduction of cells. All LV transductions were carried in presence of 8 μg/mL protamine sulfate. HEK 293T cells were cultured in Dulbecco's Modified Eagle Medium (DMEM) supplemented with 10% Hyclone Cosmic Calf serum (Thermo Fischer Scientific), 1% sodium pyruvate, non-essential aminoacids, L-Glutamine, and penicillin/streptomycin. HEK 293T cells were adapted to growth in suspension using Freestyle 293 expression media (Thermo Fischer Scientific). During passaging, LV producer cells were grown in the presence of hygromycin (150 μg/mL, Thermo Fischer Scientific), puromycin (1 μg/mL, Life Technologies Corp., Carlsbad, Calif.), blasticidin (7.5 μg/mL, Life Technologies) and zeocin (100 μg/mL, Life Technologies). HT1080 cells and H9 cells were grown in DMEM supplemented with 10% fetal bovine serum. Human CD34⁺ cells were collected from volunteers under an institutional review board-approved protocol. Human and NHP CD34⁺ were isolated, cultured and transduced as described previously. Trobridge et al., Mol Ther 18: 725-733 (2010).

Briefly, cells were transduced on CH-296 fibronectin (Takara) at 2 μg/mL in the. NHP and human CD4⁺ cells were isolated and cultured using a similar protocol to the one described previously for human CD4⁺ cells. Sather, et al., Efficient modification of CCR5 in primary human hematopoietic cells using a megaTAL nuclease and AAV donor template. Science Translational Medicine 7: 307ra156 (2015).

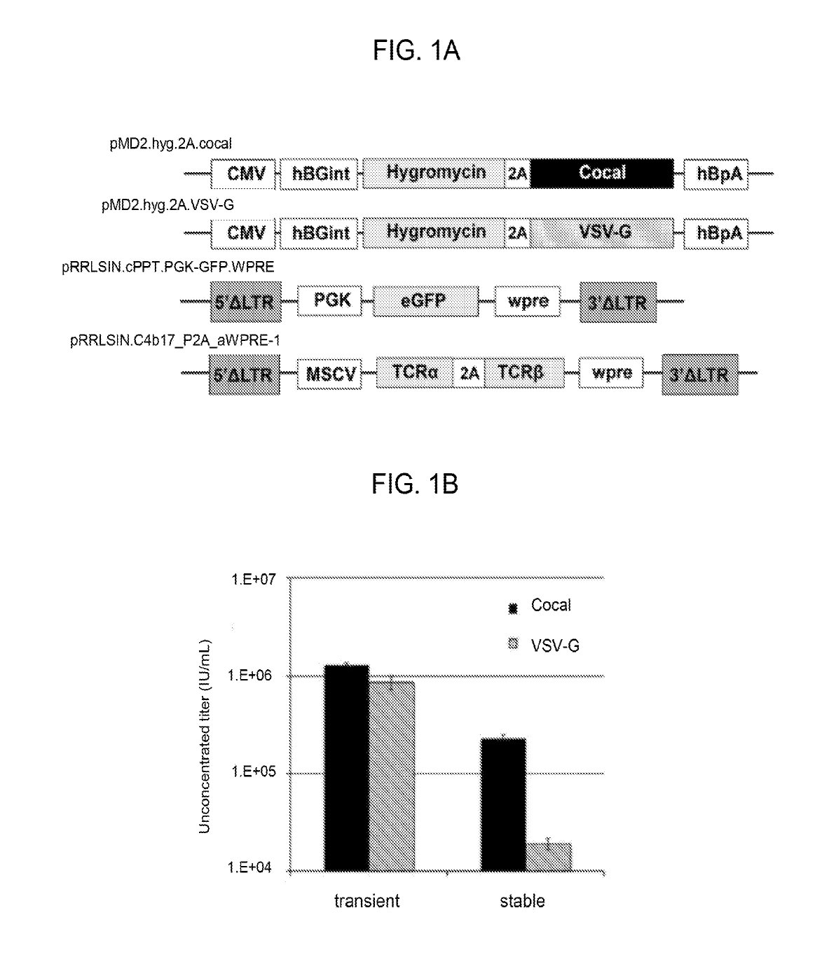

Results. Stable expression of the cocal envelope in HEK 293T cells results in the production of over 10-fold more LV as compared to VSV-G expression. Stable LV producer cells have several advantages over transient vector production. However, constitutive expression of the VSV-G envelope has previously been associated with high levels of cytotoxicity, (Ory et al., Proc Natl Acad Sci USA 93: 11400-11406 (1996)) and has largely contributed to the challenges associated with the development of high titer, stable LV producer cell lines. To determine if the cocal envelope is a better choice than VSV-G for constructing a producer cell line, either the cocal or the VSV-G envelope was stably expressed in human embryonic kidney (HEK) 293T cells, and the resulting LV titers from each cell line were measured. Plasmid pMD2.G was modified so that both the hygromycin resistance gene and the envelope encoding gene (cocal or VSV-G) were expressed from the same promoter using a 2A self-cleaving polypeptide (FIG. 1A) to ensure that hygromycin resistant cells will concomitantly express the envelope proteins. Following hygromycin selection, LV production was induced in cells stably expressing cocal or VSV-G envelope by transfection of all other required 3^{rd} generation helper plasmids and of the self-inactivating (SIN) configured LV plasmid pRRLSIN.cPPT.PGK-GFP.WPRE (FIG. 1A). This plasmid encodes the same SIN LV backbone used in multiple clinical trials of gene transfer (National clinical trials [NCT] identifiers NCT02343666 and NCT01331018) with a human phosphoglycerate kinase promoter driving expression of an enhanced green fluorescent protein (eGFP) for ease of monitoring transduction. For simplicity, this plasmid will be referred to as PGK-eGFP. LV titer was determined by transducing the HT1080 fibrosarcoma cell line and by measuring expression of the transgene in transduced cells using flow cytometry. While no significant difference in titer was found when cocal LV and VSV-G LV were produced in 293T cells by the standard protocol using transient transfection (FIG. 1B, left), stable expression of the cocal envelope in 293T cells produced 10-fold more infectious particles as compared to VSV-G expression (FIG. 1B, right). Of note, these two cell lines were developed in parallel using a similar protocol, and LV production was measured from bulk cultures, not from selected producer cell clones, explaining why the titers obtained with the cell lines are lower than titers obtained with transient transfections. Overall, stable expression of the cocal envelope results in higher LV titer as compared to VSV-G, and may be the preferred envelope for the generation of a LV producer cell lines.

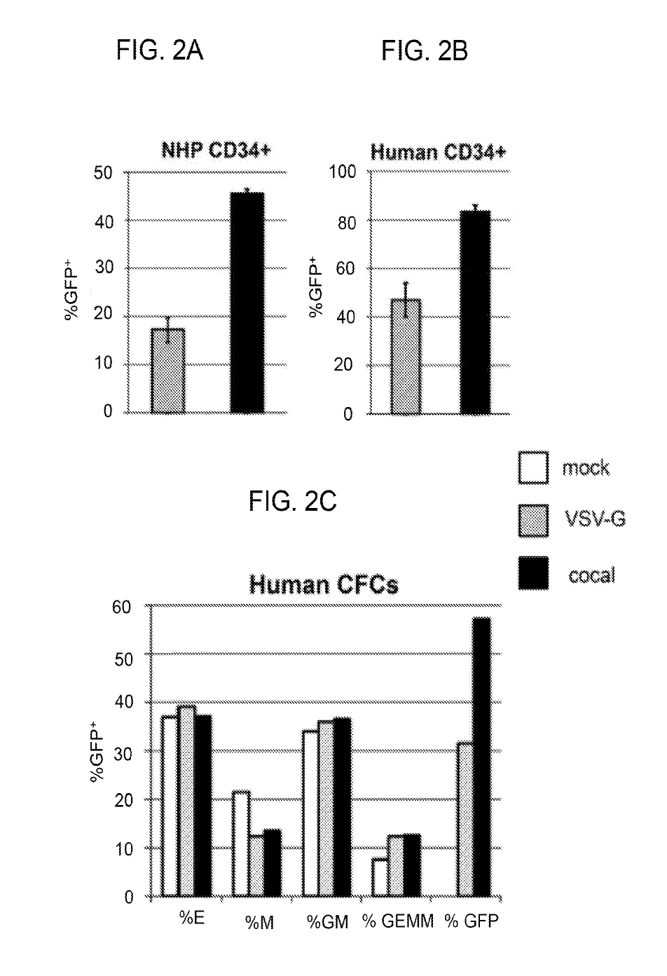

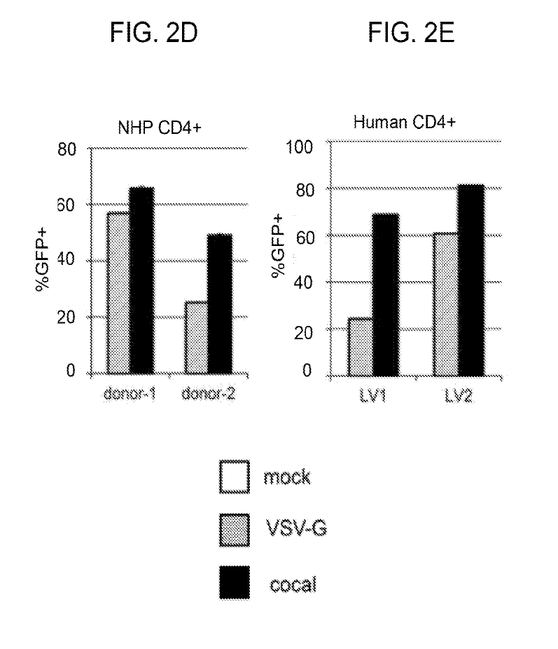

Cocal-pseudotyped LVs outperform VSV-G vectors in the transduction of human and nonhuman primate CD34⁺ and CD4⁺ cells. It has previously been demonstrated that cocal LVs have a broad tropism and efficiently transduce HSCs from different species at low multiplicities of infection (MOI). Trobridge et al., Mol Ther 18: 725-733 (2010). Here, it is demonstrated that cocal LVs transduce both nonhuman primate (NHP) and human CD34⁺ cells at higher efficiencies than VSV-G LVs when matched by multiplicity of infection (FIGS. 2A, 2B). Cocal LV transduced human CD34⁺ exhibit similar differentiation potential compared to VSV-G LV-transduced CD34⁺ cells as determined by the colony-forming assays (FIG. 2C). Extending these studies, it was also observed that cocal LVs transduced both NHP and human CD4⁺ T cells more efficiently than VSV-G LV at low MOIs (FIGS. 2D, 2E). Altogether, these results validate the use of cocal LVs for efficient gene transfer in HSCs and T cells and suggest higher potency than VSV-G LVs.

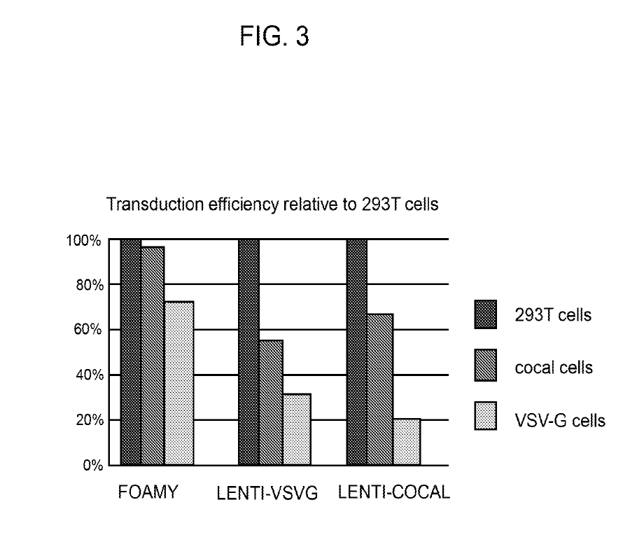

Envelope interference assay suggests overlap in receptors usage for cell entry by cocal and VSV-G LV vectors. Although the cocal envelope glycoprotein shares 71.5% identity at the amino acid level with the VSV-G envelope glycoprotein, it is not known whether the two envelopes use the same receptor(s) for cell entry. This question was addressed using the phenomenon of receptor interference. Interference occurs by blocking entry receptors of a cell when envelope proteinsare produced within the same cell. In other words, an envelope protein expressed in a cell will bind to its receptor and prevent infection by viruses that use the same cell surface molecule as receptor. Cocal-expressing cells, VSV-G-expressing cells (both described in FIG. 1B), or control 293T cells were transduced with eGFP cocal LVs, VSV-G LVs, or foamy virus vectors as control. It was found that transduction by cocal LV or VSV-G LV was inhibited 40-80% by cells expressing either the cocal or VSV-G envelope relative to control 293T cells (FIG. 3), indicating that both envelopes partially interfered with LV binding. In contrast, foamy vector transduction was not affected by cocal envelope expression, showing that interference is receptor-specific and consistent with the notion of distinct cellular receptor used by the foamy envelope. These results suggest overlap in cell entry receptor usage by cocal and VSV-G envelopes.

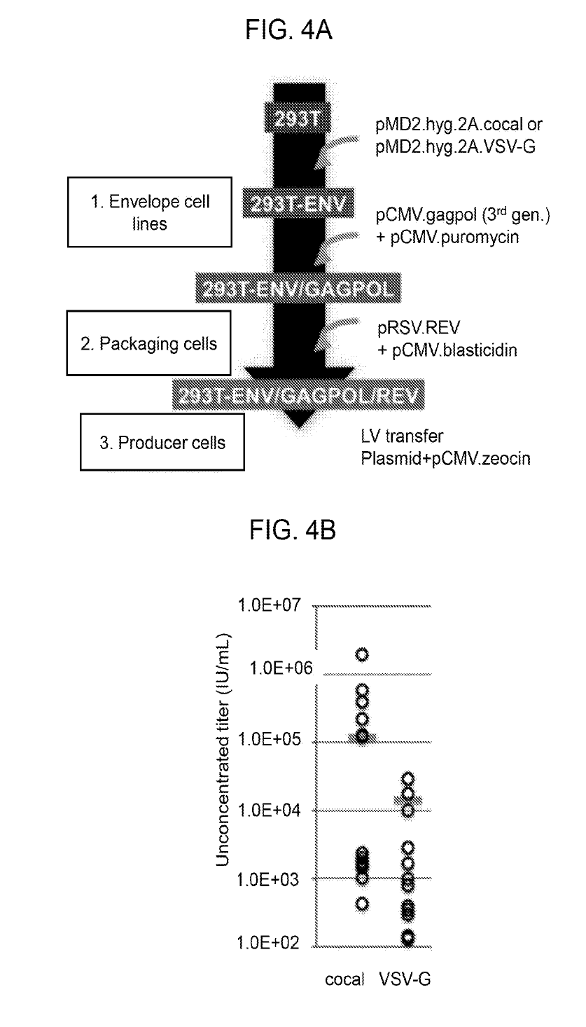

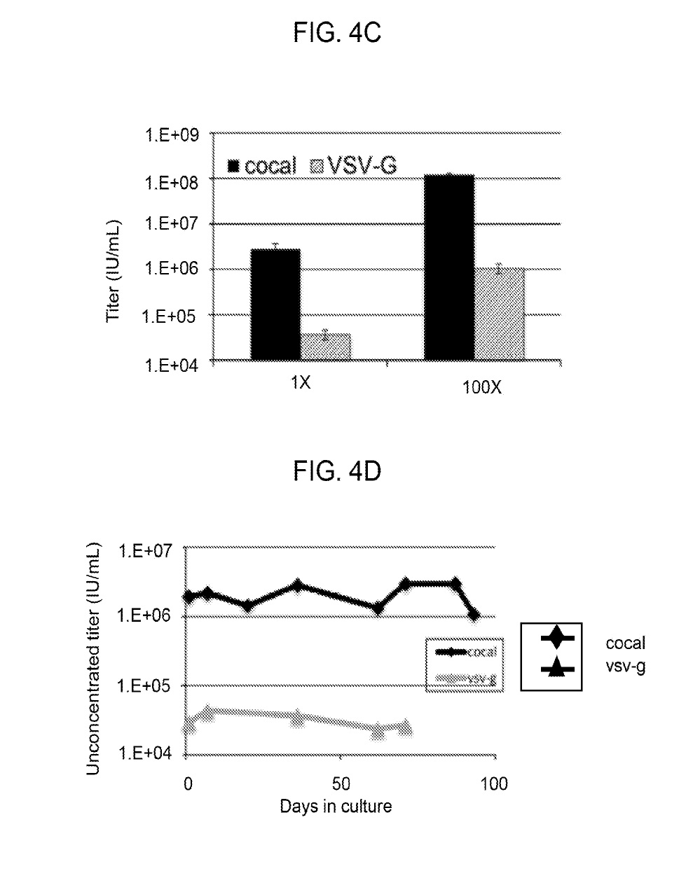

Development of cocal and VSV-G 3^{rd} generation LV packaging cell lines. The cocal or VSV-G-expressing cell lines were derived into 3^{rd} generation LV packaging cell lines by sequential transfection of plasmids encoding the gagpol and rev helper genes along with plasmids encoding antibiotic resistance genes (FIG. 4A). It was found that a molar ratio of 1:5 of selection plasmid to helper plasmid, respectively, was optimal for LV production. Stable expression of GagPol and Rev proteins was achieved by selection with puromycin and blasticidin, respectively, for 3 weeks. LV production was then induced in the resulting cell lines, now expressing all helper genes (referred to as packaging cells), by transfection of the lentiviral plasmid PGK-eGFP. As shown in FIG. 4B (red bar), bulk cocal packaging cells produced on average 8-fold more LV as compared to bulk VSV-G packaging cells (1.2×10⁵ IU/mL for cocal vs. 1.5×10⁴ IU/mL for VSV-G). To further increase vector titer, single cocal or VSV-G packaging cell clones were isolated using a limiting dilution approach. Single cells were seeded in a 96-well plate and expanded to 6-well plates under selective conditions, and the resulting LV titers produced by each clone was quantified.

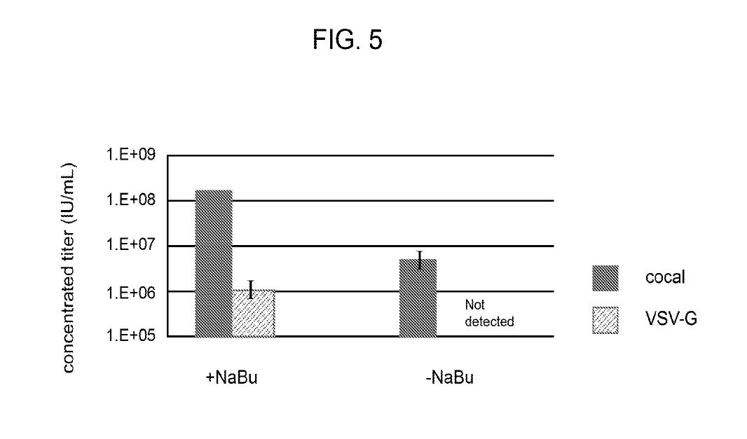

Screening of over a dozen individual clones for each line identified the best cocal packaging clone that generated unconcentrated titers of 1.9×10⁶ IU/mL and the best VSV-G packaging clone that generated 2.9×10⁴ IU/mL (FIG. 4B). Similar titers could be achieved from these best clones when scaling up culture conditions to 15-cm plates (FIG. 4C). Upon concentration by centrifugation (100×), the best cocal packaging clone produced a LV titer that averaged 1.2×10⁸ IU/mL, while the best VSV-G packaging clone produced 1.1×10⁶ IU/mL. Treatment with sodium butyrate (NaBu) during the production phase was necessary as titers were significantly lower in the absence of treatment (FIG. 5). The titer obtained with the highest performing cocal packaging cell line is equivalent to the titer routinely obtained using standard LV production protocols by transient transfection (FIG. 1B). The greatest utility of this cell line is achievement of stable vector production of equivalent titer over an extended period of time in culture. To verify the stability of these packaging cells, LV production after culture under selection for 2 to 3 months was measured, and consistent titers over time (FIG. 4D) were observed. In summary, a stable LV packaging cell line using the cocal envelope, which produces significantly higher titers than VSV-G packaging cells generated with an identical approach was successfully developed.

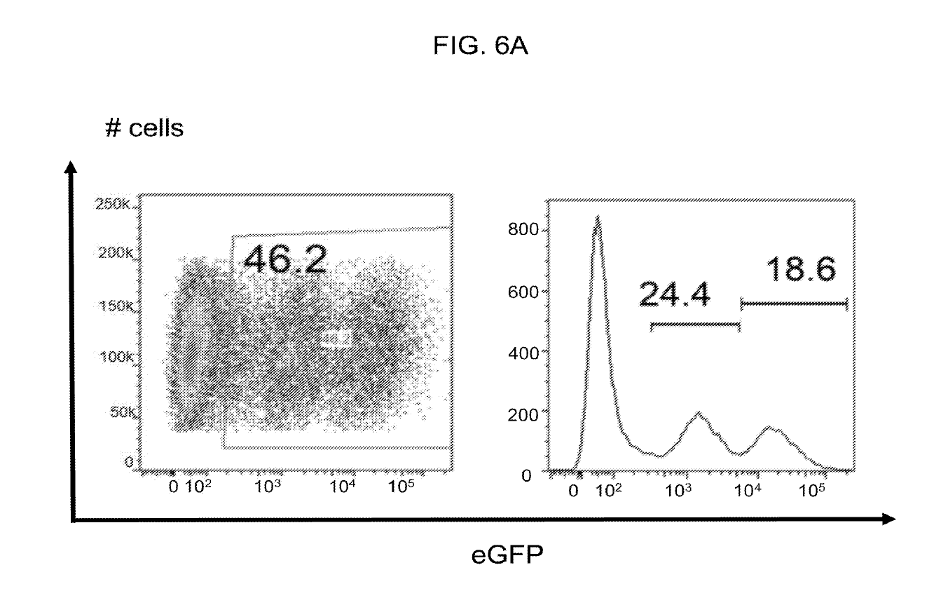

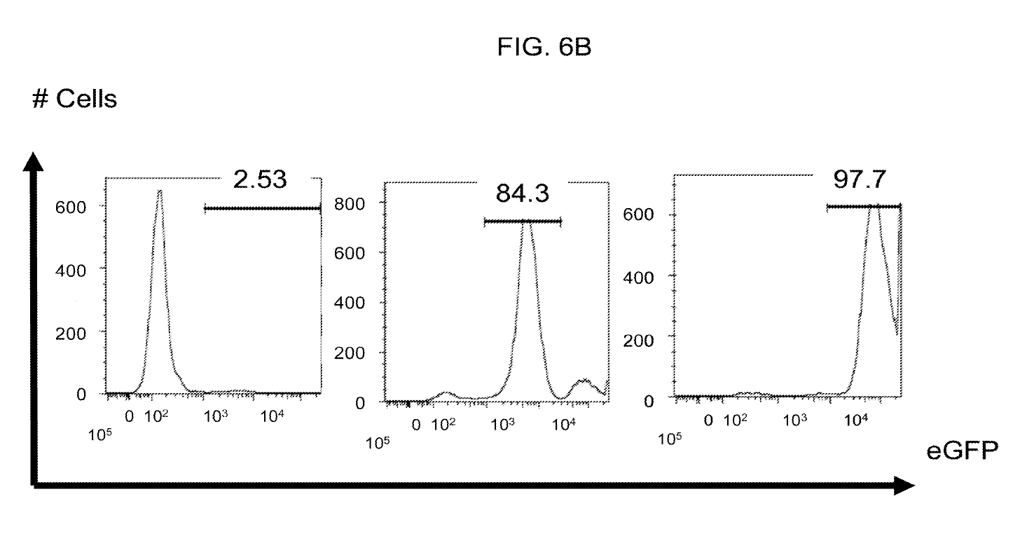

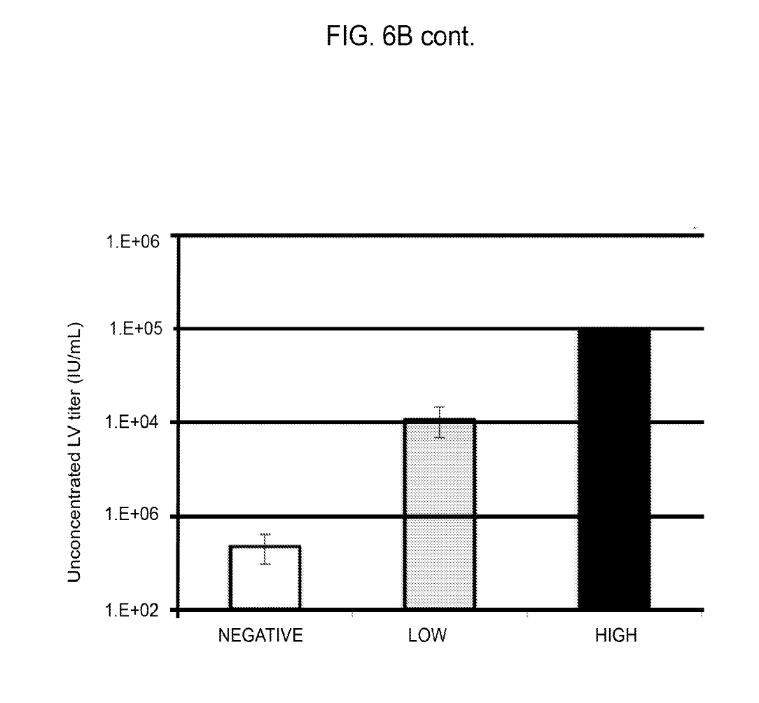

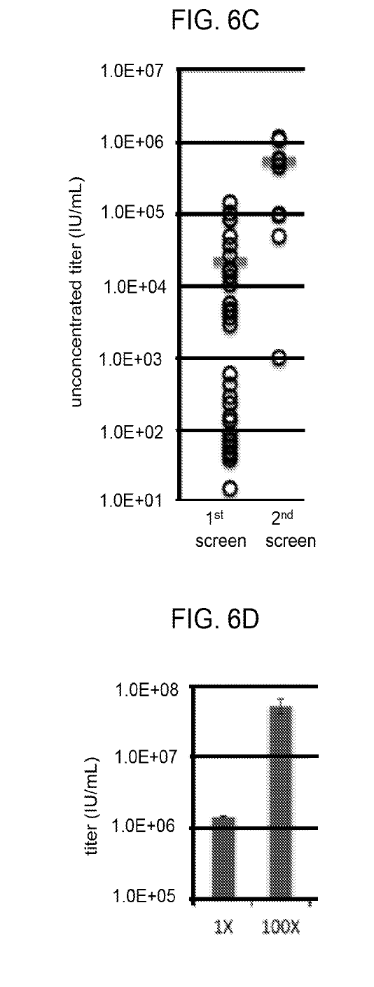

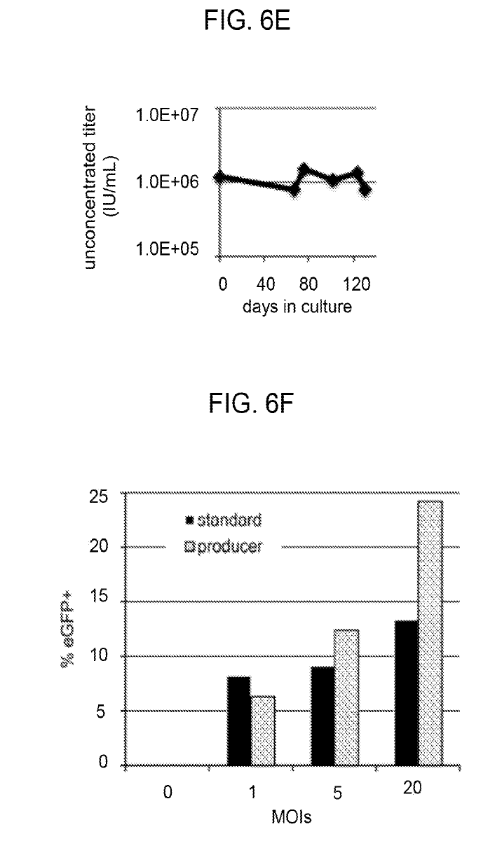

Generation of a high titer 3^{rd} generation eGFP expressing cocal SIN LV producer cell line. It was next sought to stably express lentiviral plasmids in packaging cells in order to obtain LV producer cells. Since cocal packaging cells produced a LV titer almost 100-fold higher than VSV-G packaging cells (FIG. 4C), the focus was on cocal cells to generate a LV producer cell line. As a proof of principle experiment, the PGK-eGFP lentiviral construct (FIG. 1A) was used. Cocal packaging cells were transfected with both the lentiviral construct and a plasmid encoding zeocin resistance, and selection was carried out in zeocin-containing media for 3 weeks to allow for stable expression of the plasmid. Flow cytometry analysis of the producer cells revealed that only 50% of the cells were eGFP positive, and expressed various levels of the eGFP protein (FIG. 6A). In an effort to increase vector production, first cells were sorted based on the mean fluorescence intensity of eGFP expression as assessed by flow cytometry, and LV production in low-, intermediate-, and high-eGFP+ subpopulations was analyzed (FIG. 6B, upper). A direct correlation was found between the levels of eGFP expressed by the cells and the resulting LV titer. In the high-eGFP-expressing cells, an unconcentrated titer of 1×10⁵ IU/mL was achieved while low-expressing cells produced a titer of less than 1 ×10³ IU/mL (FIG. 6B, lower). As an alternative approach to increase LV titer from producer cells, single producer clones were isolated and screened similarly to that described earlier for packaging cells. Analysis of two dozen clones identified six clones that produced LV titers greater than bulk cells (compare open circles with bar, FIG. 6C, left). LV titer in the best cocal producer clone was confirmed from culture in 15-cm plates and resulted in unconcentrated titer (1×) above 1.0×10⁶ IU/mL (±1.7×10⁴ IU/mL) and concentrated titer (100×) reaching 5.3×10⁷ IU/mL (±6.4×10⁶ IU/mL) (FIG. 6D). LV titer generated from these producer cells was stable after serial passages under selective conditions for 4 months (FIG. 6E). LV titer was further increased by 2-fold in this best producer of cocal cell clones following a second round of single clone screening (FIG. 6C, right), which should now generate concentrated titer above 10⁸ IU/mL. The performance of the cocal eGFP vector made with the producer cell line was next assessed as compared to identical cocal eGFP vector made using the standard transfection protocol. NHP CD34⁺ cells were transduced with three different multiplicities of infection (MOIs), and transduction efficiency was measured after 4 days based on the fraction of GFP+ cells. As shown in FIG. 6F, it was found that the vector generated with producer cells resulted in considerably higher transduction efficiencies at MOIs of 5 and 20 as compared to vector generated by transient transfection but transduced CD34⁺ cells equally at MOI of 1. Therefore, eGFP LV generated with the disclosed cocal producer cell line shows superior transduction efficiencies of primary cells as compared to LV made with the standard protocol.

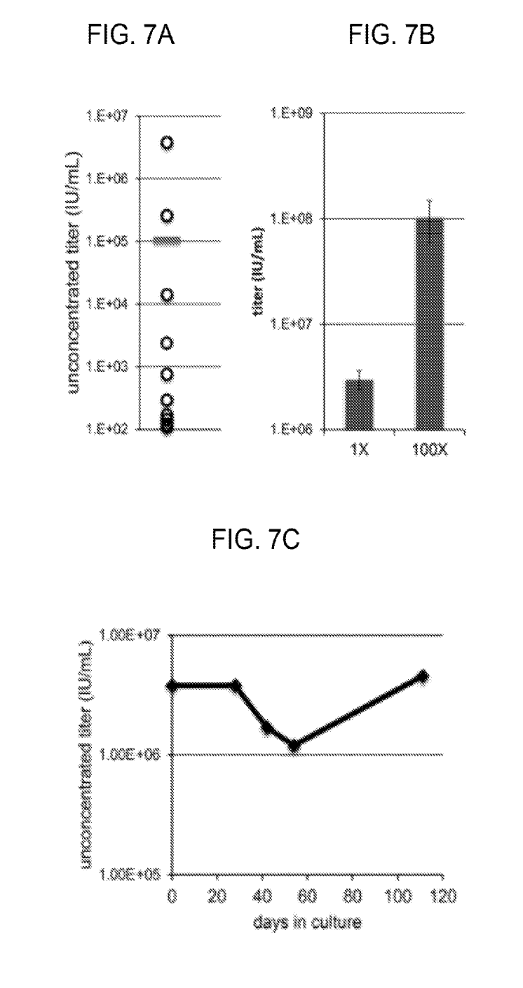

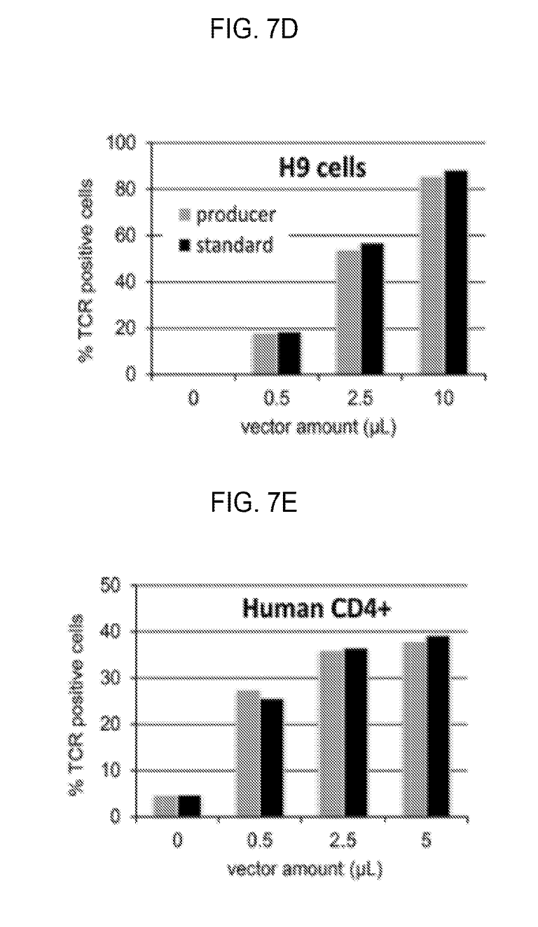

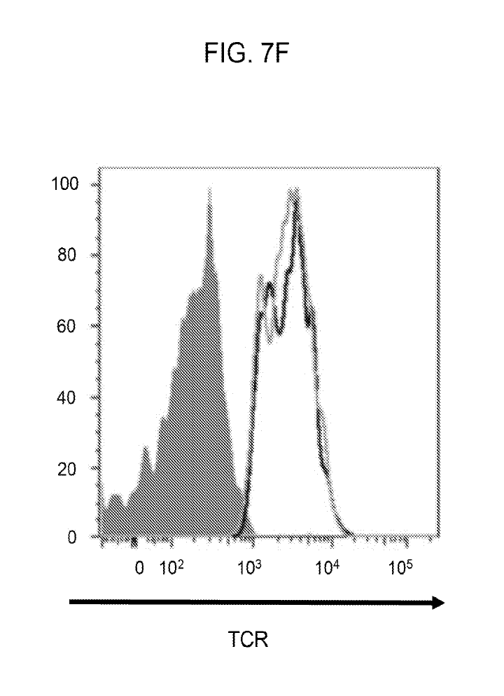

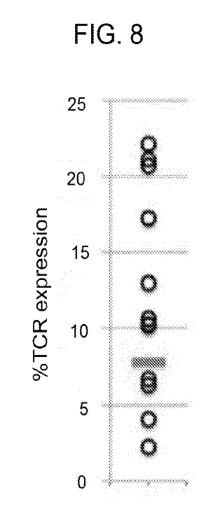

Generation of a 3^{rd} generation cocal SIN LV producer cell line encoding a T cell receptor specific for the WT1 tumor antigen. The findings that cocal LVs transduce CD4⁺ T cells at higher efficiencies than VSV-G LVs (FIGS. 2D, 2E) prompted development of a cocal producer cell line that could be used in immunotherapy applications. The LV construct, pRRLSIN.C4b17_P2A_aWPRE-1, which encodes the alpha and beta chains of an HLA-A2-restricted T cell receptor (TCR) specific for the cancer antigen WT1 (FIG. 1A) was employed. Chapuis, A G, Ragnarsson, G B, Nguyen, H N, Chaney, C N, Pufnock, J S, Schmitt, T M, et al. (2013). Transferred WT1-reactive CD8+ T cells can mediate antileukemic activity and persist in post-transplant patients. Science Translational Medicine 5: 174ra27; Schmitt, et al., Blood 122: 348-356 (2013). A similar procedure to the one described above was used for the eGFP producer cell line, starting with cocal packaging cells, with the difference that LV titer was now measured either by quantitative PCR (qPCR) or by antibody staining of surface receptor expression in T cells. After selecting cells that stably expressed the TCR lentiviral plasmid, titer in a dozen single producer cell clones was measured. Two clones produced LV titers that were higher than the bulk cell population as measured by qPCR (compare open circles with bar, FIG. 7A), and the best producer clone generated unconcentrated titer reaching 3.8×10⁶ IU/m L. Repeated rounds of LV production carried out with this TCR LV producer clone showed an average unconcentrated titer of 3.0×10⁶ IU/mL (±6.1×10⁵ IU/mL) and a concentrated titer of 1.0×10⁸ IU/mL (±4.5×10⁷ IU/mL) (FIG. 7B). In addition, LV titer generated by these cells was stable for over 100 days when cultured under selection (FIG. 7C). The performance of those LVs made by producer cells was next directly compared with the vector made with the standard protocol. Both the H9 lymphoma cell line and human CD4⁺ T cells were transduced with different amounts of each vector, and transduction efficiency was measured by TCR surface staining. For this experiment, LV preparations were used with comparable concentrated titers of 2×10⁸ IU/mL prepared either by producer cells or by transient transfection. Following transduction of H9 cells or CD4⁺ T cells, it was found that TCR expression was nearly identical between cells transduced with cocal LVs generated by producer cells versus cells transduced with LVs made with the standard protocol (FIGS. 5D, 5E). In addition, equivalent level of TCR expression was measured in CD4⁺ cells transduced with each LV, as determined by mean fluorescence intensity (FIG. 7F). These results demonstrate that cocal LV generated with t producer cell line is equivalent to LV generated with the standard protocol, even in the context of primary human T cells. Similarly to that described previously for the eGFP producer cells, LV titers were further increased by carrying out a second round of single clone screening, this time using TCR expression in H9 cells as readout. Several clones generated significantly higher LV titers than the original cell line, with an increase in titer by as much as 3-fold (FIG. 8). In conclusion, a cocal LV producer cell line was successfully developed for the WT1-specific TCR. It is capable of achieving concentrated titers above 1×10⁸ IU/mL, thus making it a suitable system for the generation of clinical-grade vector.

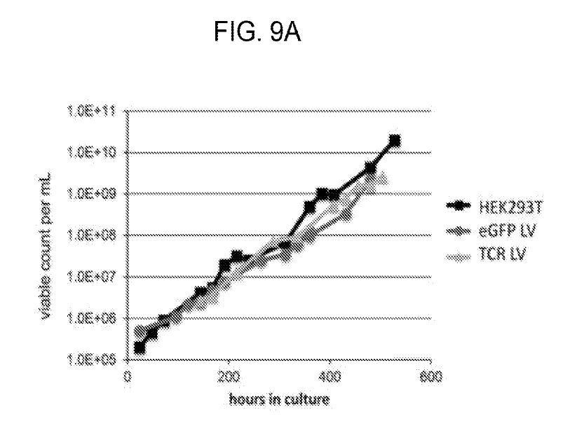

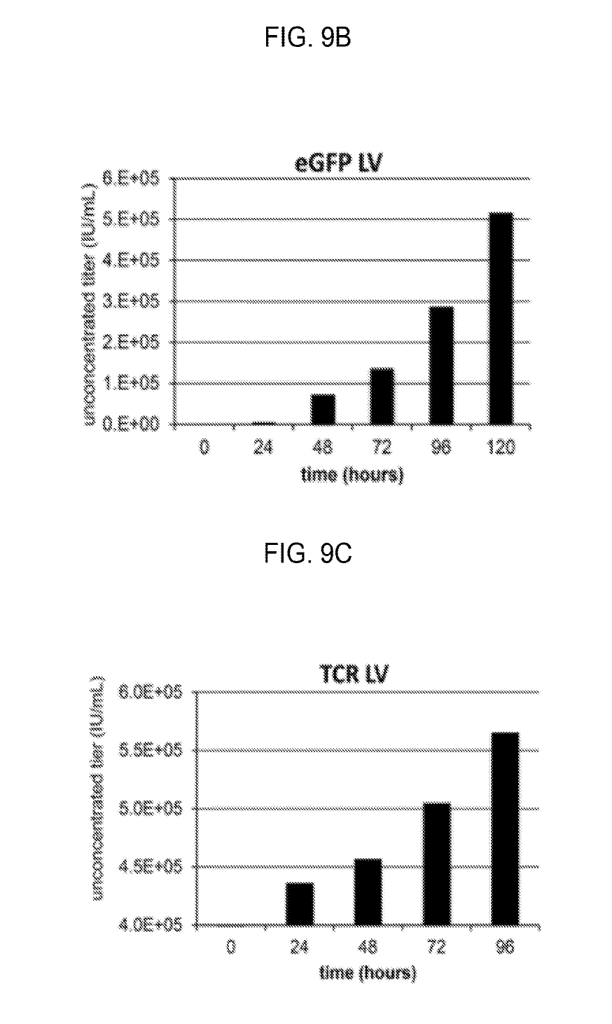

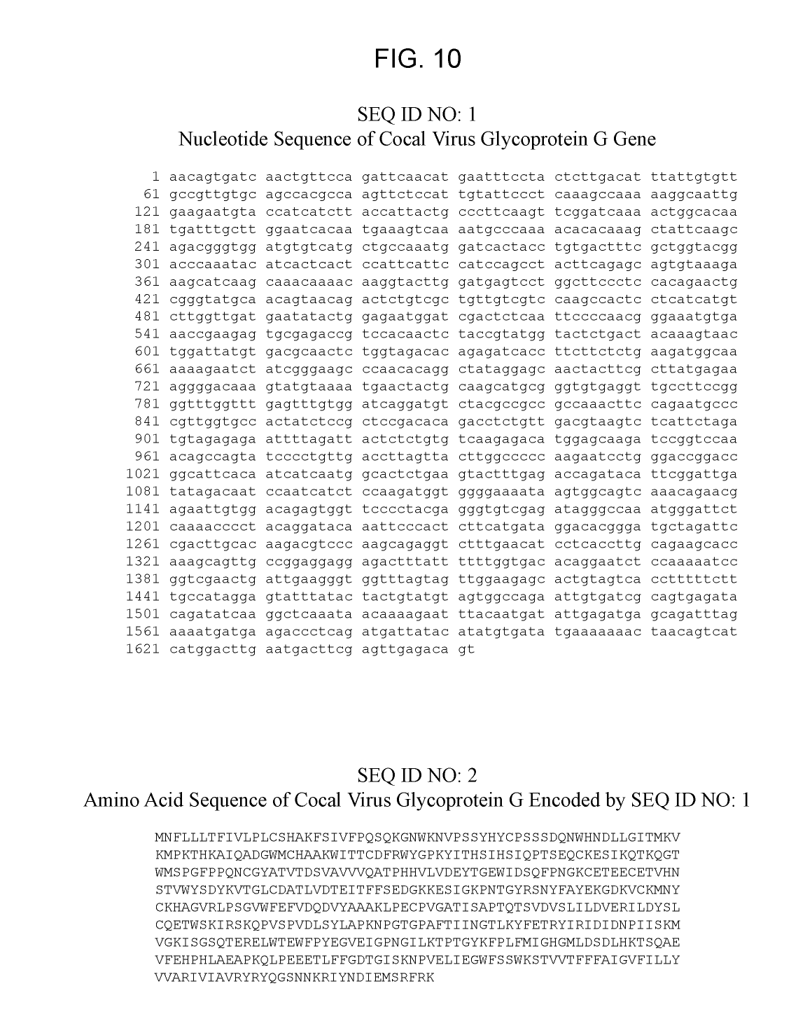

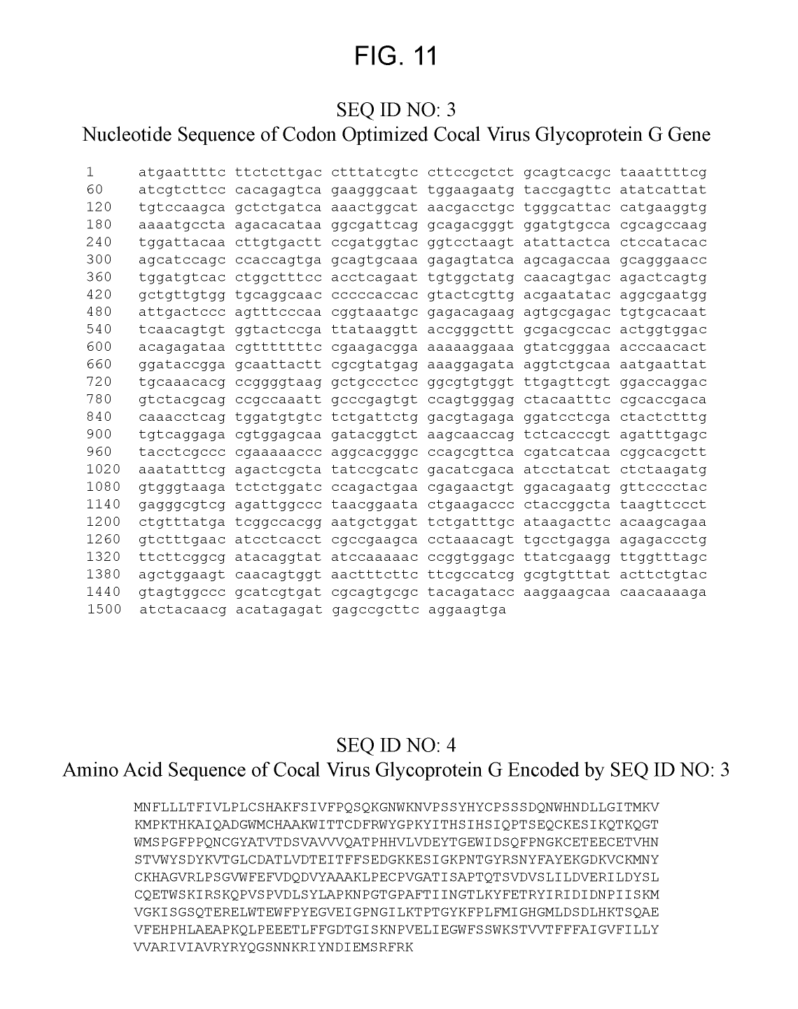

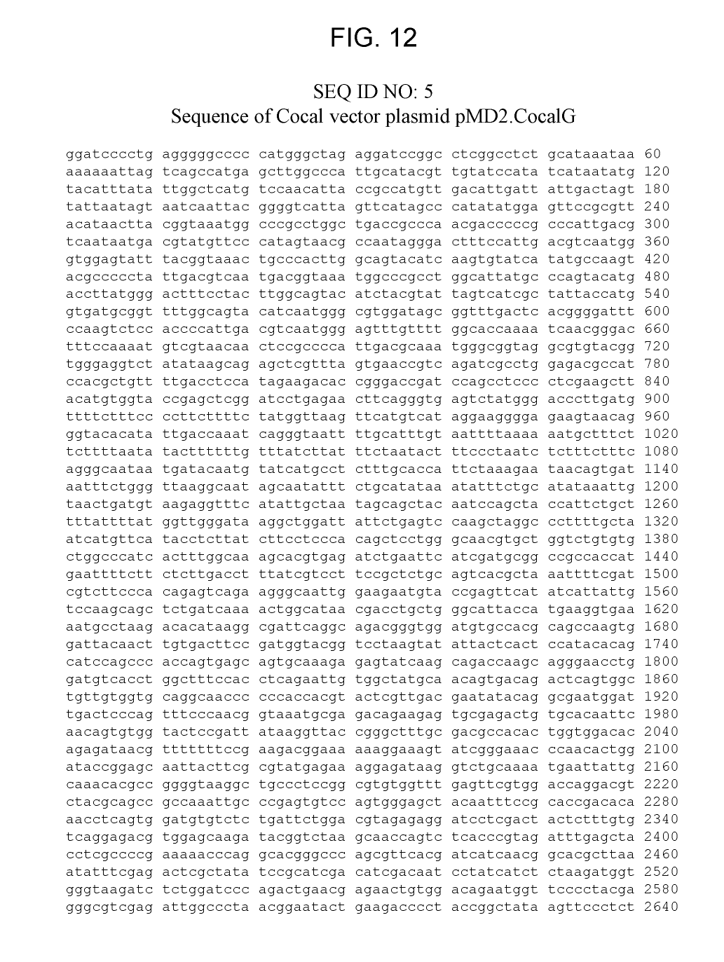

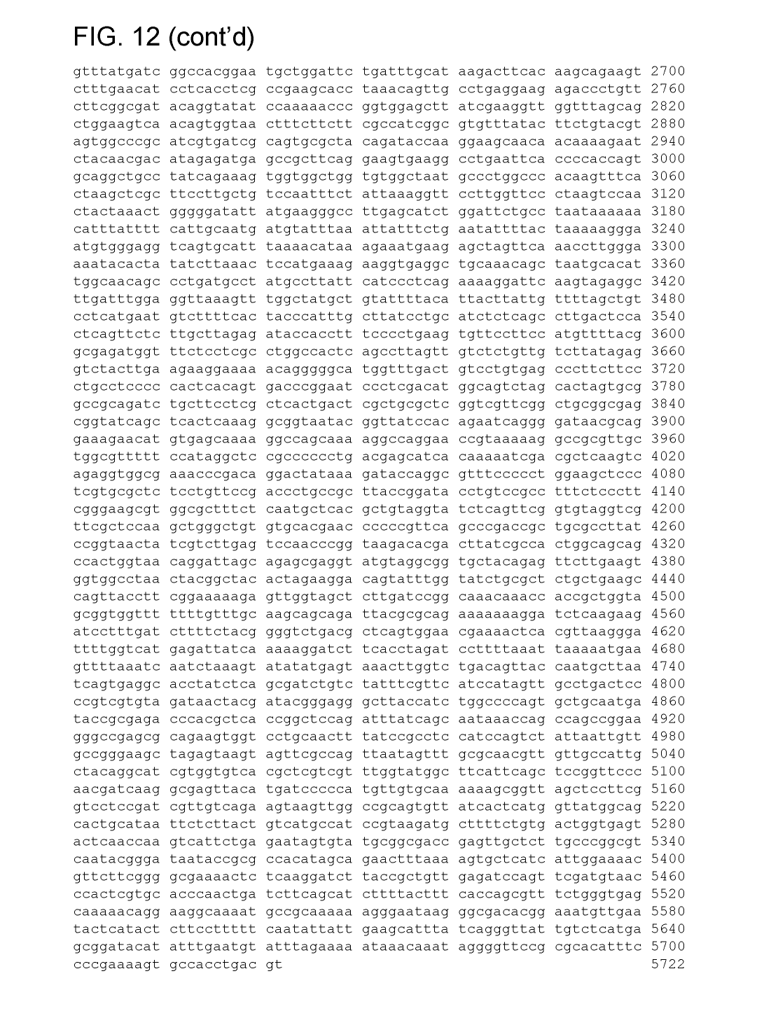

Adaptation of cocal producer cells to suspension/serum-free cultures for large-scale LV preparations. Current LV gene therapy trials often involve the treatment of a large number of patients, which, in turn, require sizeable volumes of clinical grade vector. To initiate efforts toward the scaling-up of vector production, cocal producer cells were adapted for growth in suspension/serum-free cultures. Adherent eGFP and TCR best LV producer clones characterized previously (FIG. 6D and FIG. 7B) were grown in 10-mL shaker flasks in Freestyle 293 Expression serum-free medium, with media changes performed every 3 days. After 2 weeks of culture under these conditions, both producer cell lines grew robustly in suspension cultures in the absence of selection, with a doubling time averaging 41 hours as compared to 29 hours for 293T cells (FIG. 9A). LV production was measured in these suspension producer cell lines over time and a steady increase in vector produced by eGFP producer cells over the 5 days monitored was found, reaching an unconcentrated titer of 5×10⁵ IU/mL (FIG. 9B). LV titer in the TCR producer cell line was slightly improved, reaching an unconcentrated titer of 5.5×10⁵ IU/mL by day 4 (FIG. 9C). LV production was increased to 100mL cultures using spinner bottles, then to 1 L using a bioreactor. On average, suspension cells achieved 100× concentrated titers of 2.0×10⁷ IU/mL (±2.7×10⁶ IU/mL) for eGFP LV and 4.0×10⁷ IU/mL (±5.9×10⁶ IU/mL) for TCR LV. These titers are 3- to 5-fold lower than titers obtained from adherent cells, and production will require further optimization. However, these data demonstrate the possibility of adapting the disclosed producing cell lines to a suspension culture system, which should have significant advantages for future large-scale LV production.

Supporting amino acid and nucleotide sequences not expressly disclosed herein can be readily obtained by consulting publicly-available databases. Variants of sequences obtained from the databases are also included within the scope of the disclosure. Variants of protein gene products can include one or more amino acid additions, deletions, stop positions, or substitutions, as compared to a protein disclosed herein.

An amino acid substitution can be a conservative or a non-conservative substitution. Variants of proteins disclosed herein can include those having one or more conservative amino acid substitutions. A “conservative substitution” involves a substitution found in one of the following conservative substitutions groups: Group 1: alanine (Ala or A), glycine (Gly or G), Ser, Thr; Group 2: aspartic acid (Asp or D), Glu; Group 3: asparagine (Asn or N), glutamine (Gln or Q); Group 4: Arg, lysine (Lys or K), histidine (His or H); Group 5: Ile, leucine (Leu or L), methionine (Met or M), valine (Val or V); and Group 6: Phe, Tyr, Trp.

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys; acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W. H. Freeman and Company.

Variants of protein and nucleotide sequences disclosed or referenced herein can also include those with at least 70% sequence identity, at least 80% sequence identity, at least 85% sequence, at least 90% sequence identity, at least 95% sequence identity, at least 96% sequence identity, at least 97% sequence identity, at least 98% sequence identity, or at least 99% sequence identity to a protein or nucleotide sequence disclosed or referenced herein.

“% sequence identity” refers to a relationship between two or more sequences, as determined by comparing the sequences. In the art, “identity” also means the degree of sequence relatedness between protein sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” mean any set of values or parameters which originally load with the software when first initialized.

Moreover, if a technique to practice the invention is not expressly disclosed, the technique adopted should utilize conventional methods of virology and immunology within the skill of the art, many of which are described in the following references: Fields et al., “Virology” (3^{rd} Edition, 1996); Sambrook, et al., “Molecular Cloning: A Laboratory Manual” (2^{nd} Edition, 1989); “DNA Cloning: A Practical Approach, vol. I & II” (D. Glover, ed.); “Oligonucleotide Synthesis” (N. Gait, ed., 1984); “Nucleic Acid Hybridization” (B. Hames & S. Higgins, eds., 1985); Perbal, “A Practical Guide to Molecular Cloning” (1984); Ausubel et al., “Current Protocols in Molecular Biology” (New York, John Wiley and Sons, 1987); Bonifacino et al., “Current Protocols in Cell Biology” (New York, John Wiley & Sons, 1999); Coligan et al., “Current Protocols in Immunology” (New York, John Wiley & Sons, 1999); Harlow and Lane Antibodies: a Laboratory Manual Cold Spring Harbor Laboratory (1988); and Lo, Ed., “Antibody Engineering: Methods and Protocols,” Part 1 (Humana Press, Totowa, N.J., 2004); and Gulick and Fahl, Proc. Natl. Acad. Sci. USA, 92:8140-8144 (1995).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. Within the current disclosure, a material effect would result in a statistically significant decrease in vector production by a producer cell line disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, if references have been made to patents, printed publications, journal articles and other written text throughout this specification (referenced materials herein), each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A producer cell that stably expresses a cocal envelope, retroviral helpers and a retroviral vector.

2. A producer cell of claim 1 wherein the producer cell additionally expresses a selection marker.

3. A producer cell of claim 1 wherein the producer cell additionally expresses a self-cleaving peptide.

4. A producer cell of claim 1 wherein the producer cell stably expresses the cocal envelope, retroviral helpers and retroviral vector for at least 100 days.

5. A producer cell of claim 1 wherein the producer cell stably expresses the cocal envelope, retroviral helpers and retroviral vector while in suspension.

6. A producer cell of claim 5 wherein the suspension is serum-free.

7. A producer cell of claim 6 wherein the suspension is at least 1 liter.

8. A producer cell of claim 1 wherein the retroviral vector encodes a therapeutic protein.

9. A producer cell of claim 1 wherein the producer cell is a human embryonic kidney (HEK) 293 cell.

10. A producer cell of claim 1 wherein the stable expression of the cocal envelope, retroviral helpers and retroviral vector results in production of a cocal envelope pseudotyped retroviral vector.

11. A method of producing a producer cell that stably produces a cocal envelope pseudotyped retroviral vector comprising:
Transfecting a cell with a plasmid encoding a cocal envelope, a self-cleaving peptide, and a selection marker;
Transfecting the cell with one or more helper plasmids when the cell is stably expressing the cocal envelope; and
Transfecting the cell with a retroviral plasmid when the cell is stably expressing the cocal envelope.

12. A method of claim 11 wherein the retroviral plasmid is a lentiviral plasmid.

13. A method of claim 11 wherein the producer cell is a human embryonic kidney (HEK) 293 cell.

14. A method of harvesting a cocal envelope pseudotyped retroviral vector produced by a cell of claim 1 comprising:
washing a cell of claim 1;
treating the cell with sodium butyrate;
harvesting the cell's supernatant;
filtering the supernatant; and
centrifuging the filtrate.

15. A method of claim 14 wherein sodium butyrate is replaced or supplemented with Scriptaid, APHA Compound 8, Apicidin, (−)-Depudecin, Sirtinol, or trichostatin A.
